(19) United States
(12) Patent Application Publication
(10) Pub. No.: US 2021/0322311 A1
(43) Pub. Date: Oct. 21, 2021

(54) Inhalable Formulation of a Solution Containing Tiotropium Bromide and Olodaterol

(71) Applicants: Cai Gu Huang, Shrewsbury, MA (US); Xiaoqian Wang, Shanghai (CN); Pengpeng Gu, Shanghai (CN)

(72) Inventors: Cai Gu Huang, Shrewsbury, MA (US); Xiaoqian Wang, Shanghai (CN); Pengpeng Gu, Shanghai (CN)

(73) Assignee: Cai Gu Huang, Shrewsbury, MA (US)

(21) Appl. No.: 17/231,821

(22) Filed: Apr. 15, 2021

Related U.S. Application Data
(60) Provisional application No. 63/011,867, filed on Apr. 17, 2020.
(60) Provisional application No. 63/011,224, filed on Apr. 16, 2020.
(60) Provisional application No. 63/011,220, filed on Apr. 16, 2020.

(51) Int. Cl. A61K 9/00; A61K 31/439; A61K 31/538; A61K 47/02; A61K 47/12; A61K 47/18

(52) U.S. Cl. CPC A61K 9/0078; A61K 31/439; A61K 31/538; A61K 9/0073; A61K 47/12; A61K 47/186; A61K 47/183; A61K 47/02

(57) ABSTRACT

The present invention relates to a liquid, propellant-free pharmaceutical preparation and a method for administering the pharmaceutical preparation by nebulizing the pharmaceutical preparation in an inhaler. The propellant-free pharmaceutical preparation comprises a solvent, Tiotropium or a pharmaceutically acceptable salt thereof, Olodaterol or a pharmaceutically acceptable salt thereof, a pH adjusting agent, and a pharmacologically acceptable additive.

### PRIORITY STATEMENT

This application claims the benefit of the filing date of U.S. Provisional Patent Application Nos. 63/011,867, filed on Apr. 17, 2020; 63/011,224, filed on Apr. 16, 2020; and 63/011,220, filed on Apr. 16, 2020, the contents of which are incorporated herein by reference in its entirety.

### BACKGROUND OF THE INVENTION

Tiotropium bromide monohydrate is chemically described as (1a, 2β, 4β, 5α, 7β)-7-[(Hydroxydi-2-thienylacetyl)oxy]-9,9-dimethyl-3-oxa-9-azoniatricyclo [3.3.1.02,4] nonane bromide monohydrate, and has the following chemical structure:

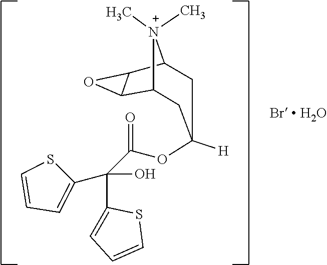

Olodaterol hydrochloride, is chemically described as 2H-1,4-Benzoxazin-3H(4H)-one, 6-hydroxy-8-[(1R)-1-hydroxy-2-[[2-(4-methoxyphenyl)-1, 1-dim ethyl ethyl]-amino] ethyl]-, monohydrochloride, is disclosed in U.S. Pat. Nos. 7,220,742, 7,491,719, 7,056,916, 7,727,984, and has the following chemical structure:

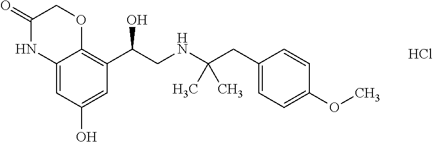 [SMILES: COc1ccc(CC(C)(C)NC[C@H](O)c2cc(O)cc3c2OCC(=O)N3)cc1 ; Cl]

Tiotropium is a long-acting, muscarinic antagonist which is often referred to as an anticholinergic. It has a similar affinity to muscarinic receptor subtypes M1 to M5. In the airways, it exhibits a pharmacological effect by inhibiting M3-receptors on smooth muscle, leading to bronchodilation. The competitive and reversible nature of antagonism was shown with human and animal origin receptors and isolated organ preparations.

Olodaterol is a long-acting beta-2-adrenergic agonist (LABA) that activates beta-2 adrenoreceptors on airway smooth muscle, causing bronchodilation. Beta-2 receptors are the adrenergic receptors in bronchial smooth muscle. These two compounds have valuable pharmacological properties. Tiotropium and Olodaterol can provide therapeutic benefit in the treatment of asthma or chronic obstructive pulmonary disease, including chronic bronchitis and emphysema.

The present invention relates to a propellant-free inhalable formulation of Tiotropium and Olodaterol, or pharmaceutically acceptable salts thereof, dissolved in water, in conjunction with inactive ingredients preferably administered using a nebulization inhalation device, and the propellant-free inhalable aerosols resulting therefrom. The pharmaceutical formulations disclosed in the current invention are especially suitable for nebulization inhalation, which has much better lung deposition (typically up to 55-60%), compared to drying powder inhalation.

The pharmaceutical formulations of the present invention are particularly suitable for administering the active substances by nebulization inhalation, especially for treating asthma and chronic obstructive pulmonary disease.

### SUMMARY OF THE INVENTION

The present invention relates to pharmaceutical formulations of Tiotropium and Olodaterol and their pharmaceutically acceptable salts or solvates which can be administered by nebulization inhalation. The pharmaceutical formulations according to the invention meet high quality standards.

One aspect of the present invention is to provide an aqueous pharmaceutical formulation containing Tiotropium and Olodaterol, or pharmaceutically acceptable salts thereof, which meet the high standards needed in order to achieve optimum nebulization of the formulation using the inhalers mentioned hereinbefore. Pharmaceutical stability of the active substances in the formulation should be a storage time of some years, preferably one year, more preferably three years.

Another aspect is to provide propellant-free formulations that are solutions containing Tiotropium and Olodaterol, or pharmaceutically acceptable salts thereof, which can be nebulized under pressure using an inhaler, which preferably is a nebulization inhaler device, to provide an aerosol, wherein the particle size of the aerosol falls reproducibly within a specified range.

Another aspect of the invention is to provide pharmaceutical formulations that are solutions comprising Tiotropium and Olodaterol, or pharmaceutically acceptable salts thereof, and other inactive excipients which can be administered by nebulization inhalation using ultra-sonic based or air pressure based nebulizers/inhalers. The pharmaceutical formulations exhibit a stability suitable to allow a storage time of few months or years, preferably 1-6 months, more preferably one year, and most preferably three years.

More specifically, another aspect is to provide a stable pharmaceutical formulation that is an aqueous solution containing Tiotropium and Olodaterol, or pharmaceutically acceptable salts thereof, and other excipients which can be administered by nebulization inhalation using an ultrasonic, jet, or mesh nebulizers. The inventive formulations have substantial long term stability. In one embodiment, the formulations have a storage time of at least about 6-24 months at a temperature of from about 15° C. to about 25° C.

### DETAILED DESCRIPTION OF THE INVENTION

It is advantageous to administer a liquid formulation without propellant gases, using suitable inhalers, in order to achieve a better distribution of active substances in the lung. Furthermore, it is very important to increase the lung deposition of a drug being delivered by inhalation.

Currently, traditional pMDI or DPI (drying powder inhalation) devices only deliver about 20-30% of a drug into the lung, resulting in a significant amount of drug being deposited on the month and throat, which can enter the stomach and cause unwanted side effects and or secondary absorption through the oral digestion system.

Therefore, there is a need in the art to improve drug delivery by inhalation so as to significantly increase lung deposition.

The pharmaceutical formulation of the invention is a solution that is converted into an aerosol destined for the lungs by the nebulizer. The pharmaceutical solution is sprayed with the nebulizer by high pressure.

Nebulization devices useful for administering the pharmaceutical formulations of the present invention are those in which an amount of less than about 8 milliliters of pharmaceutical solution can be nebulized in one puff, preferably less than about 2 milliliters, most preferably less than about 1 milliliter, so that the inhalable part of aerosol corresponds to a therapeutically effective quantity. The average particle size of the aerosol formed from one puff is less than about 15 microns, preferably less than about 10 microns. The solution formulations must not contain any ingredients which might interact with the inhaler and affect the pharmaceutical quality of the solution or of the aerosol produced. In addition, the active substances in the pharmaceutical formulations are very stable when stored and can be administered directly.

Therefore, one aspect of the present invention is to provide an aqueous pharmaceutical formulation containing Tiotropium and Olodaterol, or pharmaceutically acceptable salts thereof, which meet the high standards needed in order to be able to achieve optimum nebulization of the solution using the inhalers mentioned hereinbefore. Preferably the active substances in the pharmaceutical formulation are stable, and have a storage time of some years, preferably one year, more preferably three years.

Another aspect of the current invention is to provide propellant-free formulations that are solutions containing Tiotropium and Olodaterol, or pharmaceutically acceptable salts thereof, which are nebulized under pressure using an inhaler, preferable a nebulization inhaler, wherein the pharmaceutical composition delivered by the produced aerosol falls reproducibly within a specified range for particle size.

Another aspect is to provide an aqueous pharmaceutical formulation that is a solution containing Tiotropium and Olodaterol, or pharmaceutically acceptable salts thereof, and other inactive excipients which can be administered by inhalation.

According to the invention, any pharmaceutically acceptable salts or solvates of Tiotropium and Olodaterol may be used for the formulation. When the term Tiotropium or and Olodaterol is used within the scope of the present invention, it is to be taken as a reference to Tiotropium or a salt or solvate thereof and Olodaterol or a salt or a solvate thereof, respectively.

In one embodiment the salt of Tiotropium is Tiotropium bromide monohydrate and the salt of Olodaterol is Olodaterol hydrochloride.

In one embodiment, the active substances are a combination of Tiotropium bromide monohydrate and Olodaterol hydrochloride.

In the formulations according to the invention, Tiotropium and Olodaterol are dissolved in a solvent. In one embodiment, the solvent is water.

The concentration of the Tiotropium and Olodaterol in the finished pharmaceutical preparation depends on the therapeutic effect desired. In one embodiment, the concentration of Olodaterol in the formulation is between about 18.2 μg/100 ml and about 182 mg/100 ml, for example, about 25 mg/100 ml. In one embodiment, the concentration of Tiotropium is between about 20.7 μg/100 ml and about 207 mg/100 ml, for example, about 28 mg/100 ml.

In the formulations according to the invention, if desired, the pH can be adjusted by adding an acid or base, to the formulation as a pH adjusting agent. In one embodiment, hydrochloric acid and/or sodium hydroxide is added as the pH adjusting agent.

Other comparable pH adjusting agents can be used in the present invention. An example of a suitable pH adjusting agent is citric acid and/or its salts.

The pH is selected to maintain stability of the active ingredients. In one embodiment, the pH ranges from about 1.0 to about 5.0, for example, from about 2.0 to about 3.5.

In the formulations according to the invention, if desired, a stabilizer or complexing agent can be included in the formulation. Suitable stabilizers or complexing agents include, but are not limited to, edetic acid (EDTA) or one of the known salts thereof, disodium edetate, or edetate disodium dihydrate. In one embodiment, the formulation contains edetic acid and/or a salt thereof.

Other comparable stabilizers or complexing agents can be used in the present invention. Other stabilizers or complexing agents include, but are not limited to, citric acid, edetate disodium, and edetate disodium dihydrate.

The phrase “complexing agent,” as used herein, means a molecule which is capable of entering into complex bonds. In one embodiment, these compounds have the effect of complexing cations. In one embodiment, the concentration of the stabilizer or complexing agent is from about 1 mg/100 ml to about 500 mg/100 ml, for example, from about 10 mg/100 ml to about 200 mg/100 ml. In one embodiment, the complexing agent is edetate disodium dihydrate in a concentration of from about 1 mg/100 ml to about 500 mg/100 ml.

In the formulations according to the invention, if desired, the isosmotic status of the formulation can be adjusted by adding an isosmotic adjusting agent, such as sodium chloride. In one embodiment, the formulation contains sodium chloride.

In one embodiment, the quantity of sodium chloride is from about 0.8% (w/v) to about 1.0% (w/v), for example, about 0.9% (w/v).

In one embodiment, the Oldaterol or salt thereof is Oldaterol hydrochloride, wherein a dose of Olodaterol hydrochloride is from about 3 μg to about 80 preferably from about 3 μg to about 50 and more preferably from about 5 μg to about 30 and the Tiotropium or a salt thereof is Tiotropium bromide monohydrate, wherein a dose of Tiotropium bromide monohydrate is from about 3 μg to about 80 preferably about 3 μg to about 50 and more preferably from about 5 μg to about 30 μg.

In one embodiment of the formulations, the Tiotropium bromide and Olodaterol are present in solution.

It is advantageous if all the ingredients of the formulation are present in solution when the formulation is administered using an inhaler.

The phrase “additives,” as sued herein means any pharmacologically acceptable and therapeutically useful substance which is not an active substance, but can be formulated together with the active substances in a pharmacologically suitable solvent, in order to improve the qualities of the formulation. Preferably, these substances have no appreciable pharmacological effects or, at least, no undesirable pharmacological effects in the context of the desired therapy.

Suitable additives include, but are not limited to, other stabilizers, complexing agents, antioxidants, surfactants, and/or preservatives which prolong the shelf life of the finished pharmaceutical formulation, vitamins, and/or other additives known in the art.

The pharmaceutical formulation is converted in the nebulizer into aerosol that is destined for the lungs. The pharmaceutical solution is sprayed with the nebulizer by high pressure.

### EXAMPLES

Materials and Reagents:
Tiotropium bromide monohydrate, from Anovent Pharmaceutical Co., Ltd. in Nanchang, China.
Olodaterol hydrochloride, from Kalulan Science & Technology Co., Ltd. in Shanghai, China.
Sodium chloride, from Merck.
Citric acid, from Merck.
Sodium hydroxide, from Titan Reagents Co., Ltd. in Shanghai, China.
Hydrochloric acid, from Titan Reagents Co., Ltd. in Shanghai, China. 50% benzalkonium chloride (refered to BAC) aqueous solution is commercially available and may be purchased from Spectrum Pharmaceuticals Inc.
Edetate disodium dehydrate is commercially available and may be purchased from purchased from Merck & Co.

### Example 1

The preparation of sample I, sample II, and sample III inhalation solutions is as follows: active and inactive ingredients according to the amounts provided in Table 1, were dissolved in 90 ml of purified water and the resulting solution then adjusted to the target pH with hydrochloric acid or sodium hydroxide. Purified water was then added to final volume of 100 ml.

TABLE 1
Ingredient Contents of Sample I, Sample II and Sample
III of 100 ml of an Inhalable Formulation

| | | | |
|---|---|---|---|
| Ingredients | Sample I | Sample II | Sample III |

| | | | | | | |
|---|---|---|---|---|---|---|
| Olodaterol hydrochloride | 18.2 | μg | 1.82 | mg | 182 | mg |
| Tiotropium bromide | 20.7 | μg | 2.07 | mg | 207 | mg |
| monohydrate | | | | | | |
| Sodium chloride | 0.8 | g | 0.9 | g | 1.0 | g |

| | | | |
|---|---|---|---|
| hydrochloric acid or | To pH 1.0 | To pH 3.0 | To pH 5.0 |
| sodium hydroxide | | | |
| Purified water | Added to | Added to | Added to |
| | 100 ml | 100 ml | 100 ml |

### Example 2

The preparation of sample IV inhalation solution is as follows: active and inactive ingredients according to the amounts provided in table 2, were dissolved in 90 ml purified water and the resulting solution then adjusted to the target pH with hydrochloric acid or sodium hydroxide. Purified water was then added to final volume of 100 ml.

TABLE 2
Ingredient Contents of Sample IV of 100 ml Inhalable Formulation

| Ingredients | Sample IV | |
|---|---|---|
| Olodaterol hydrochloride | 0.547 | mg |
| Tiotropium bromide monohydrate | 0.620 | mg |
| Sodium chloride | 0.9 | g |

| | |
|---|---|
| Hydrochloric acid | To pH 2.9 |
| Purified water | Added to 100 ml |

### Example 3

Sample IV was sprayed using a nebulization inhaler. Malvern Spraytec (STP5311) was used to measure the particle size of the resulting droplets. The particle size distribution is provided in Table 3.

TABLE 3
Particle Size Distribution of Sample
IV by Using Nebulization Inhaler

| | |
|---|---|
| Sample Number | Droplet size (μm) |

| | | |
|---|---|---|
| Sample IV | D10 | 1.74 |
| | D50 | 4.89 |
| | D90 | 11.79 |

### Example 4

TABLE 4
Osmotic Pressure of Sample IV

| Sample Number | Osmotic pressure |
|---|---|
| Sample IV | 290 mOsm |

### Example 5

Influence of pH on Stability:

Stability is highly dependent on pH. Six samples were prepared according to Table 5. Olodaterol hydrochloride (referred to as OH) and Tiotropium bromide monohydrate (referred to as TB) in the amounts provided in Table 5 were dissolved in 40 ml of purified water. The pH of samples 1-5 were then adjusted to pH 2.0, 2.5, 3.0, 3.5, 4.0 with HCl, respectively. Sample 6 pH was left unadjusted (pH is 6.3). The resulting mixtures were then sonicated until the components completely dissolved. Purified water was then added to a final volume of 50 ml.

The formula of the six samples is shown in Table 5. Each Sample was stored at 60° C. for 28 days. Experimental data for the stability of each sample is provided in Tables 6-8.

TABLE 5
Formulation Design of TB and OH Screening at Different pH Values

| Ingredients | Sample 1 | | Sample 2 | | Sample 3 | | Sample 4 | | Sample 5 | | Sample 6 | |
|---|---|---|---|---|---|---|---|---|---|---|---|---|
| TB | 14 | mg | 14 | mg | 14 | mg | 14 | mg | 14 | mg | 14 | mg |
| OH | 12.5 | mg | 12.5 | mg | 12.5 | mg | 12.5 | mg | 12.5 | mg | 12.5 | mg |
| HCl | Adjust to | | Adjust to | | Adjust to | | Adjust to | | Adjust to | | pH not | |
| | pH 2.0 | | pH 2.5 | | pH 3.0 | | pH 3.5 | | pH 4.0 | | adjusted | |
| Purified | 50 | ml | 50 | ml | 50 | ml | 50 | ml | 50 | ml | 50 | ml |
| water | | | | | | | | | | | | |

Impurity A, CAS number:4746-63-8

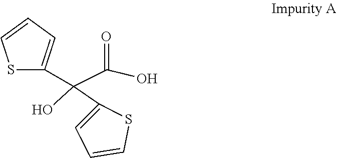 [SMILES: O=C(O)C(O)(c1cccs1)c1cccs1]

Impurity F, CAS number: 704-38-1

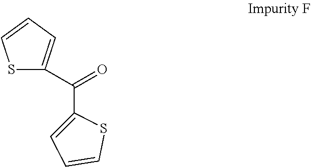 [SMILES: O=C(c1cccs1)c1cccs1]

Impurity OLO-14:

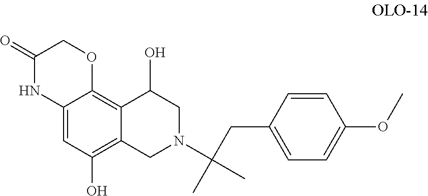 [SMILES: COc1ccc(CC(C)(C)N2Cc3c(O)cc4c(c3C(O)C2)OCC(=O)N4)cc1]

TABLE 6
Stability at Different pH Values(Conditions: 0 day)

| Impurity | | RRT | Sample 1 | Sample 2 | Sample 3 | Sample 4 | Sample 5 | Sample 6 |
|---|---|---|---|---|---|---|---|---|
| TB | Impurity A | 0.86 | ND | ND | ND | ND | 0.06 | 0.53 |
| OH | OLO-14 | 1.30 | ND | ND | ND | ND | ND | ND |

| | | | | | | |
|---|---|---|---|---|---|---|
| Total impurity of TB | No report | No report | No report | No report | No report | 0.53 |
| Total impurity of OH | No report | No report | No report | No report | No report | No report |
| total impurity of TB and | No report | No report | No report | No report | No report | 0.53 |
| OH (Ignore less than 0.1%) | | | | | | |

ND: Not detected

TABLE 7
Stability at Different pH Values (Conditions: 60° C., 7days, 75% RH)

| Impurity | | RRT | Sample 1 | Sample 2 | Sample 3 | Sample 4 | Sample 5 | Sample 6 |
|---|---|---|---|---|---|---|---|---|
| TB | Impurity A | 0.86 | 0.12 | 0.11 | 0.31 | 1.49 | 9.32 | 9.01 |
| | Impurity F | 1.85 | 0.07 | 0.04 | 0.06 | 0.15 | 0.35 | 0.26 |
| OH | OH-4 | 0.64 | 0.17 | 0.1 | 0.11 | 0.08 | 0.09 | ND |
| | Unknown | 0.69 | 0.03 | 0.02 | 0.04 | 0.04 | 0.02 | ND |
| | impurity 1 | | | | | | | |
| | Unknown | 1.26 | 0.13 | 0.02 | ND | ND | ND | ND |
| | impurity 2 | | | | | | | |
| | Unknown | 1.28 | 0.23 | 0.09 | 0.06 | 0.12 | 0.09 | ND |
| | impurity 3 | | | | | | | |
| | Unknown | 1.33 | 0.19 | 0.03 | ND | ND | ND | ND |
| | impurity 4 | | | | | | | |
| | OLO-14 | 1.35 | ND | 0.02 | 0.03 | 0.09 | 0.15 | 0.07 |

| | | | | | | |
|---|---|---|---|---|---|---|
| Total impurity of TB | 0.12 | 0.11 | 0.31 | 1.64 | 9.67 | 9.27 |
| Total impurity of OH | 0.72 | 0.1 | 0.11 | 0.27 | 0.15 | 0.26 |
| Total impurity of TB and | 0.84 | 0.21 | 0.42 | 1.91 | 9.82 | 9.53 |
| OH (Ignore less than 0.1%) | | | | | | |

TABLE 8
Stability at Different pH Values(Conditions: 60° C., 14 days, 75% RH)

| Impurity | | RRT | Sample 1 | Sample 2 | Sample 3 | Sample 4 | Sample 5 | Sample 6 |
|---|---|---|---|---|---|---|---|---|
| TB | Impurity A | 0.861 | 0.16 | 0.17 | 0.56 | 2.59 | 14.3 | 21.91 |
| | Impurity F | 1.844 | 0.27 | 0.16 | 0.22 | 0.54 | 0.88 | 1.25 |
| OH | OH-4 | 0.64 | 0.27 | 0.23 | 0.19 | 0.14 | 0.13 | 0.11 |
| | Unknown | 0.69 | 0.08 | 0.06 | 0.09 | 0.08 | 0.02 | ND |
| | impurity 1 | | | | | | | |
| | Unknown | 1.263 | 0.1 | ND | ND | ND | ND | ND |
| | impurity 2 | | | | | | | |
| | Unknown | 1.29 | 0.37 | 0.12 | 0.1 | 0.09 | ND | ND |
| | impurity 3 | | | | | | | |
| | Unknown | 1.33 | 0.47 | 0.16 | 0.1 | ND | ND | ND |
| | impurity 4 | | | | | | | |
| | OLO-14 | 1.36 | 0.07 | 0.08 | 0.11 | 0.2 | 0.24 | 0.25 |

| | | | | | | |
|---|---|---|---|---|---|---|
| Total impurity of TB | 0.43 | 0.33 | 0.78 | 3.13 | 15.18 | 23.16 |
| Total impurity of OH | 1.32 | 0.51 | 0.5 | 0.34 | 0.37 | 0.36 |
| Total impurity of TB and | 1.75 | 0.84 | 1.28 | 3.47 | 15.55 | 23.52 |
| OH (Ignore less than 0.1%)) | | | | | | |

TABLE 9
Stability at Different pH Values(Conditions: 60° C., 28 days, 75% RH)

| Impurity | | RRT | Sample 1 | Sample 2 | Sample 3 | Sample 4 | Sample 5 | Sample 6 |
|---|---|---|---|---|---|---|---|---|
| TB | Impurity A | 0.688 | 0.09 | 0.13 | 0.66 | 4.49 | 15.63 | 14.55 |
| | Impurity F | 1.882 | 0.56 | 0.38 | 0.67 | 1.82 | 2.81 | 2.88 |
| OH | OH-4 | 0.644 | 0.42 | 0.43 | 0.32 | 0.37 | 0.23 | 0.18 |
| | Unknown impurity 1 | 0.66 | 0.11 | 0.15 | 0.19 | 0.21 | 0.1 | 0.11 |
| | Unknown impurity 2 | 1.227 | 0.07 | ND | 0.02 | ND | ND | ND |
| | Unknown impurity 3 | 1.341 | 0.08 | 0.05 | ND | 0.03 | 0.01 | ND |
| | Unknown impurity 4 | 1.367 | 0.13 | 0.07 | 0.04 | 0.05 | 0.03 | ND |
| | OLO-14 | 1.393 | 0.09 | 0.17 | 0.2 | 0.45 | 0.35 | 0.33 |
| | Unknown impurity 5 | 1.406 | 0.13 | 0.08 | ND | ND | ND | ND |

| | | | | | | |
|---|---|---|---|---|---|---|
| Total impurity of TB | 0.56 | 0.51 | 1.33 | 6.31 | 18.44 | 17.43 |
| Total impurity of OH | 0.79 | 0.75 | 0.71 | 1.14 | 0.68 | 0.62 |
| Total impurity of TB and | 1.35 | 1.26 | 2.04 | 7.45 | 19.12 | 18.05 |
| OH (Ignore less than 0.1%) | | | | | | |

As can be seen from Tables 6-9, the OH and TB containing solution is stable at pH 2.0 to 3.5, the OH and TB solution is most stable at pH 2.0 to 3.0.

### Example 6

Aerodynamic Particle Size Distribution:

50% benzalkonium chloride aqueous solution (referred to 50% BAC) and edetate disodium dihydrate according to the amounts provided in Table 10 were dissolved in 900 ml of purified water. The pH was adjusted to pH 3.4 with HCl. OH and TB according to the amounts provided in Table 10 were added to the solution and the resulting mixture sonicated until completely dissolved. Purified water was then added to a final volume of 1000 ml.

TABLE 10
Ingredient Contents of Sample 7

| Ingredient | Sample 7 | |
|---|---|---|
| TB | 0.28 | g |
| OH | 0.25 | g |
| 50% BAC | 0.2 | g |
| EDTA | 0.11 | g |
| HCl | 2.95 | |
| Purified water | 1000 | ml |

The aerodynamic particle size distribution was determined using an Andersen Scale Impactor (ACI). The inhalation device was purchased from Boehringer Ingelheim Pharmaceutical Co., Ltd., named STIOLTO RESPIMAT. The STIOLTO RESPIMAT inhaler was held close to the ACI inlet until no aerosol was visible. The flow rate of the ACI was set to 28.3 L/minute and was operated under ambient temperature and a relative humidity (RH) of 90%.

The solution of sample 7 was discharged into the ACI. Fractions of the dose were deposited at different stages of the ACI, in accordance with the particle size of the fraction. Each fraction was washed from the stage and analyzed using HPLC.

The results are provided in Table 11 below.

TABLE 11
Aerodynamic Particle Size Distribution of OH and TB Inhalation
Formulation Sample 7 Administered by Respimat Inhalation

| | TB | | OH | | |
|---|---|---|---|---|---|
| | | Percentage | | Percentage | Cut-off |
| | Dosage | content at | Dosage | content at | diameter |
| Deposited | μg | all levels | μg | all levels | μm |
| Throat | 0.5868 | 18.08% | 0.5269 | 18.74% | |
| Stage 0 | 0.2080 | 6.41% | 0.1879 | 6.68% | 9 |
| Stage 1 | 0.3591 | 11.06% | 0.3140 | 11.17% | 5.8 |
| Stage 2 | 0.3538 | 10.90% | 0.3109 | 11.06% | 4.7 |
| Stage 3 | 0.5769 | 17.77% | 0.4999 | 17.78% | 3.3 |
| Stage 4 | 0.2718 | 8.37% | 0.2719 | 9.67% | 2.1 |
| Stage 5 | 0.0736 | 2.27% | 0.0657 | 2.34% | 1.1 |
| Stage 6 | 0.1226 | 3.78% | 0.1099 | 3.91% | 0.7 |
| Stage 7 | 0.2848 | 8.78% | 0.2517 | 8.95% | 0.4 |
| Stage F | 0.4084 | 12.58% | 0.2734 | 9.72% | |
| Theoretical | 3.094 | | 2.7625 | | |
| dose(μg) | | | | | |
| Actual test | 3.2458 | | 2.8121 | | |
| dose(μg) | | | | | |
| Recovery | 104.9% | | 101.8% | | |
| rate % | | | | | |
| Fine Particle | 64.45% | | 63.42% | | |
| Fraction | | | | | |
| (FPF) | | | | | |

The larger the FPF value, the higher the atomization efficiency.

The above results confirmed that the formulation of the present invention has a good atomization effect.

### Example 7

Stability Experiment:

50% benzalkonium chloride aqueous solution (referred to 50% BAC) and edetate disodium dihydrate according to the amounts provided in Table 12 were dissolved in 4500 ml of purified water. The pH was adjusted to pH 2.85 with HCl. TB and OH according to the amounts provided in Table 12 were added to the solution and the resulting mixture sonicated until completely dissolved. Purified water was then added to a final volume of 5000 ml.

TABLE 12
Ingredient Contents of Sample 8

| Ingredients | Sample 8 | |
|---|---|---|
| TB | 1.4 | g |
| OH | 1.25 | g |
| 50% BAC | 1 | g |
| EDTA | 0.55 | g |
| HCl | 2.85 | |
| Purified water | 5000 | ml |

Sample 8 was maintained at 40° C./75% RH for 0, 1, 3, 6 months. The results are provided in Table 13 below.

TABLE 13
The Stability Results of Sample 8 (Conditions: 40°
C. ± 2° C./75% ± 5% RH)

| | 0 Month | 1 Month | 3 Months | 6 Months |
|---|---|---|---|---|
| appearance | Clarified | Clarified | Clarified | Clarified |
| | solution | solution | solution | solution |
| Impurity A of TB | ND | 0.3 | 0.4 | 1.13 |
| Impurity F of TB | ND | ND | ND | 0.06 |
| OH-4 | ND | ND | ND | 0.03 |
| OLO-14 | ND | ND | 0.12 | 0.25 |
| Total impurities | No report | 0.3 | 0.52 | 1.38 |
| TB content % | 100.96 | 100.36 | 100.82 | 98.36 |
| OH content % | 100.74 | 100.52 | 102.9 | 100.76 |
| EDTA content % | 99.68 | 98.87 | 100.48 | 103.06 |
| BAC content % | 100.67 | 102.89 | 100.95 | 102.46 |

As shown in Table 13, at pH 2.85 the TB and OH solution showed good stability. The TB and OH solution was stable for about 6 months at 40° C.±2° C./75%±5% RH.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A propellant-free inhalation formulation comprising Tiotropium or a salt or solvate thereof, Olodaterol or a salt or solvate thereof, a pH adjusting agent, and a pharmacologically acceptable additive, dissolved in a solvent.

2. The formulation of claim 1, wherein the Tiotropium or a salt or solvate thereof is Tiotropium bromide monohydrate and the Olodaterol or a salt or solvate thereof is Olodaterol hydrochloride.

3. The formulation of claim 2, wherein the Olodaterol hydrochloride is present in an amount ranging from about 182 μg/100 ml to about 182 mg/100 ml.

4. The formulation of claim 2, wherein the Tiotropium bromide is present in an amount ranging from about 20.7 μg/100 ml to about 207 mg/100 ml.

5. The formulation of claim 1, wherein the pharmacologically acceptable additive is an isosmotic adjusting agent selected from the group consisting of sodium chloride, glucose, mannitol, glucitol, and mixtures thereof.

6. The formulation of claim 5, wherein the isosmotic adjusting agent is present in an amount ranging from about 0.8% (w/w) to about 1% (w/w).

7. The formulation of claim 1, wherein the solvent is water.

8. The formulation of claim 1, wherein the pH adjusting agent is selected from the group consisting of citric acid-citrate, hydrochloric acid, citric acid, and sodium hydroxide.

9. The formulation of claim 1, wherein the formulation has a pH ranging from about 2.0 to about 3.5.

10. The formulation of claim 1, wherein the pharmacologically acceptable additive is selected from the group consisting of edetic acid, edetate disodium dihydrate, edetate disodium, citric acid, and combinations thereof.

11. The formulation of claim 1, wherein the pharmacologically acceptable additive is present in an amount ranging from about 1 mg/100 ml to about 500 mg/100 ml.

12. The formulation of claim 1, wherein the pharmacologically acceptable additive is selected from the group consisting of benzalkonium chloride, benzoic acid, sodium benzoate, and a combinations thereof.

13. The formulation of claim 1, wherein the formulation has an osmotic pressure ranging from about 100 mOsm to about 400 mOsm.

14. A method of treating asthma or COPD in a patient, comprising administering to the patient a therapeutically effective amount of the pharmaceutical formulation according to claim 1 by inhalation.

15. The method of claim 14, wherein the Olodaterol or a salt thereof is Olodaterol hydrochloride and the Tiotropium or a salt thereof is Tiotropium bromide monohydrate, wherein the Olodaterol hydrochloride is administered at a dose ranging from about 3 μg to about 80 and the Tiotropium bromide monohydrate is administered at a dose ranging from about 3 μg to about 80 μg.

16. The method of claim 15, wherein the dose of Olodaterol hydrochloride ranges from about 3 μg to about 50 μg and the dose of Tiotropium bromide monohydrate ranges from about 3 μg to about 50 μg.

17. The method of claim 14, wherein the pharmaceutical formulation is administered using a nebulization inhalation device to provide an inhalable aerosol of the pharmaceutical formulation.

18. The method of claim 17, wherein the nebulization inhalation device administers a therapeutically effective amount of the pharmaceutical formulation by aerosolizing less than about 8 milliliters of the pharmaceutical solution.

19. The method of claim 17, wherein the inhalable aerosol has a D50 that is less than about 10 μm.

20. The method of claim 17, wherein the inhalable aerosol has an average particle size of less than about 15 microns.
